(19) United States
(12) Patent Application Publication
(10) Pub. No.: US 2024/0398225 A1
(43) Pub. Date: Dec. 5, 2024

(54) DEVICE, SYSTEM AND COMPUTER-IMPLEMENTED METHOD TO ASSESS AN OCULAR MOTILITY OF A SUBJECT

(71) Applicant: Essilor International, Charenton-Le-Pont (FR)

(72) Inventors: Martha HERNANDEZ-CASTANEDA, Paris (FR); Gildas MARIN, Paris (FR); Thomas CHARBONNEAU, Paris (FR)

(73) Assignee: Essilor International, Charenton-Le-Pont (FR)

(21) Appl. No.: 18/707,037

(22) PCT Filed: Nov. 17, 2022
(86) PCT No.: PCT/EP2022/082203

§ 371 Date: May 2, 2024

(30) Foreign Application Priority Data
Nov. 25, 2021 (EP) 21306636.8

(51) Int. Cl. A61B 3/113; A61B 3/00

(52) U.S. Cl. CPC A61B 3/113; A61B 3/0033; A61B 3/0058; A61B 3/0091; A61B 2560/0223

(57) ABSTRACT

A device to assess an ocular motility of a subject, the device having at least one screen configured to display a first image to a first eye of the subject and a second image to a second eye of the subject, the first image comprising a first element that is fixed and the second image comprising a second element that moves according to a movement of the subject.

### FIELD

Various aspects of this disclosure generally relate to the field of optometry, both in the field of measurement methods but also in the field of instruments and tools for optometrists. More precisely this disclosure is related to methods and devices to assess an ocular motility of a subject.

### BACKGROUND

This disclosure is related to devices to realize a complete measure of ocular motility and binocular testing. The device of this disclosure could be for example a headset used in ergonomic conditions.

Each eye of a subject has 3 pairs of muscles, which work together to turn their eyes to point them in the same direction. It is mainly thanks to these ocular muscles that we can read without difficulty and explore our environment in an efficient and comfortable manner. These 6 muscles are controlled by 3 nerves (III, IV & VI). The term ocular motility refers to the study of these six extraocular muscles and their impact on eye movement.

Examination of ocular motility is an essential step in a complete visual examination. The good realization of this measure can help the examiner to detect problems of binocular vision such as phorias, tropias, convergence troubles or neurological troubles (for example paralysis), which causes discomfort, fatigue, etc.

Currently, there are devices to detect and measure ocular motility disorders. However, these devices are performing very basic tests or complicated tests which need a long and/or complicated explanation to be performed.

There is a need for a new type of devices and methods to realize complete tests that are accurate, fast and easy to use for examiners and subjects.

### SUMMARY

The following presents a simplified summary in order to provide a basic understanding of various aspects of this disclosure. This summary is not an extensive overview of all contemplated aspects, and is intended to neither identify key or critical elements of all aspects nor delineate the scope of any or all aspects. The sole purpose is to present some concepts of one or more aspects in a simplified form as a prelude to the more detailed description that is presented later.

One aspect of this disclosure is a device to assess an ocular motility of a subject, the device comprising at least one screen configured to display a first image to a first eye of the subject and a second image to a second eye of the subject, the first image comprising a first element that is fixed and the second image comprising a second element that moves according to a movement of the subject.

One other aspect of this disclosure is a system to assess an ocular motility of a subject comprising the device and a calculation module comprising a memory and a processor, the system or the calculation module being configured to display the first element on the first image, display the second element on the second image and to move this second element according to the movement of the part of the subject, receive a selection by the subject; once the selection is received, store a localisation of the second element in the second image and possibly a localisation of the first element in the first image.

In an embodiment of the system, the device is a virtual reality headset.

In an embodiment of the system, the calculation module is a computer or a mobile device linked to the device or being integrated into the device.

On other aspect of the disclosure is a computer-implemented method to assess an ocular motility of a subject, the method comprising the steps of displaying to a first eye of the subject a first image comprising a first element, displaying to a second eye of the subject a second image comprising a second element and moving the second element according to a movement of a part of the subject, receiving a selection by the subject, once the selection is received, storing a localisation of the second element in the second image and possibly a localisation of the first element in the first image.

### DESCRIPTION OF THE DRAWINGS

For a more complete understanding of the description provided herein and the advantages thereof, reference is now made to the brief descriptions below, taken in connection with the accompanying drawings and detailed description, wherein like reference numerals represent like parts.

FIG. 1 represents the system.

FIG. 2 represents the optical device.

FIG. 3 represents in a different way the optical device.

FIG. 4 represents a Hess-Weiss grid as seen in the optical device.

FIG. 5 represents a mismatch between the vergence and the accommodation.

FIG. 6 represents a Voronoi diagram.

FIG. 7 illustrates the difficulties arising when the background is constituted of a plurality of equidistant dots.

FIG. 8 represents the images seen by the subject.

FIG. 9 illustrates a calibration procedure of an eye tracker.

### DETAILED DESCRIPTION OF EMBODIMENTS

The detailed description set forth below in connection with the appended drawings is intended as a description of various possible embodiments and is not intended to represent the only embodiments in which the concepts described herein may be practiced. The detailed description includes specific details for the purpose of providing a thorough understanding of various concepts. However, it will be apparent to those skilled in the art that these concepts may be practiced without these specific details. In some instances, well-known structures and components are shown in block diagram form in order to avoid obscuring such concepts.

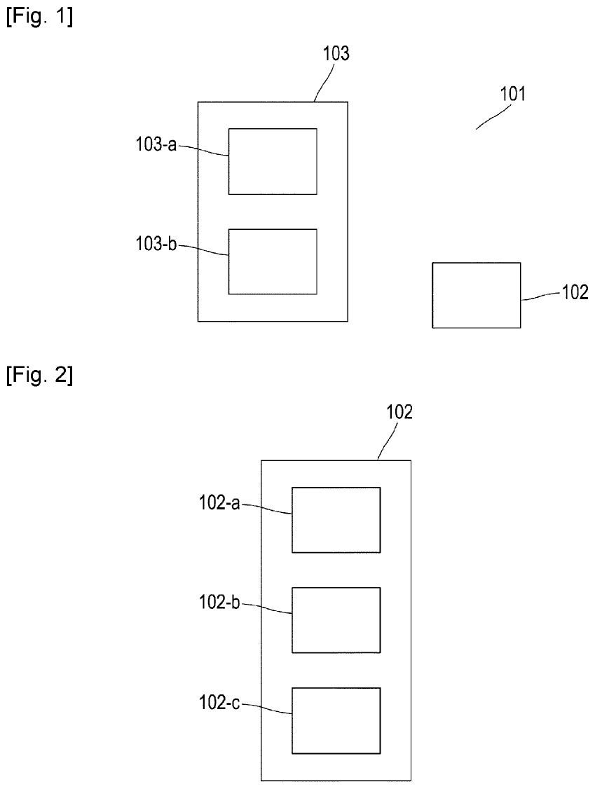

FIG. 1 represents a system 101 to assess an ocular motility of a subject. This system 101 comprises a device 102 for example an optical device 102 and a calculation module 103. The calculation module 103 comprises a memory 103-a and a processor 103-b.

Examples of processors 103-b include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure.

The memory 103-a is computer-readable media. By way of example, and not limitation, such computer-readable media may include a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by the calculation module 103.

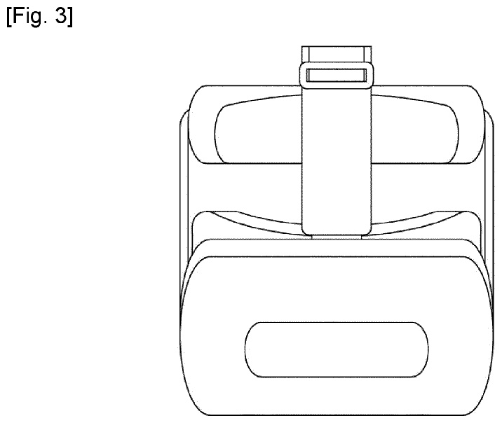

FIG. 2 represents an embodiment of the optical device 102. The optical device 102 comprises one screen 102-a and means 102-b to detect a movement of a part of the subject. The screen 102-a is configured to display a first image to a first eye of the subject (for example the right eye) and a second image to a second eye of the subject (for example the left eye). The first image comprises a first element that is fixed or still and the second image comprises a second element that moves according to a movement of the subject. The optical device can also comprise selection means 102-c configured to be activated by the subject.

In an embodiment, the optical device 102 comprises two screens, one of the two screens being configured to display the first image and the other one of the two screens being configured to display the second image.

In an embodiment, the optical device 102 comprises a single screen 102-a and a filter, for example, a polarizing filter, the single screen 102-a and the filter being configured to display the first image to the first eye and the second image to the second eye. For example, the polarizing filter comprises two parts, each part having a different polarization angle. The screen 102-a displays two images, one adapted to the polarization angle of the first part of the polarizing filter and another adapted to the polarization angle of the second part of the polarizing filter. To realize the display of the two images by the screen 102-a, a first part of the screen 102-a, associated to the first image, is polarized as the first part of the polarizing filter (the first part of the screen has the same polarizing angle as the first part of the polarizing filter) and a second part of the screen 102-a, associated with the second image, is polarized as the second part of the polarizing filter. Using this embodiment each eye is then associated with one part of the polarizer, seeing only the part of the screen having the same polarization. The two parts of the screen 102-a can be interlaced (1 line out of 2 is dedicated to the first part, and if a line is dedicated to the first part the adjacent lines are dedicated to the second part.)

The optical device 102 is configured to move the second element according to the detected movement of the part of the subject.

In an embodiment, the optical device 102 is a virtual reality headset.

In an embodiment, the calculation module 103 is a computer or a mobile device. The calculation module 103 can be linked to the optical device 102 or can be integrated in the optical device 102.

The FIG. 3 represents an embodiment of the optical device 102 implemented as a virtual reality headset. Preferentially this virtual reality headset can comprise an eye tracker with a frequency of 120 Hz (60 Hz by eye). The virtual reality headset has for example a field of view of 110° and comprises two screens with each a resolution of 1440×1600 pixels and refresh rate of 90 Hz. The eye tracker is an example of means 102-b to detect the movement and will be described more precisely below.

The optical device 102 allows to get three major informations:
Head positions and rotations (head angles)
Depth of the motility grid (vergences)
Targeted positions on the grid (with or without eye tracker)

The head positions and rotations precisely allow the realisation of the Bielschowsky test more accurately.

This system 101 allows the realization of tests aiming to assess an ocular motility of a subject. Prior to the realization of this test the pupil distance of the subject can be measured with a pupillometer.

During the test, the subject can be installed on a chair in a seated position (It could also be in a stand up position). Advantageously the subject is well positioned in order for the headset to be seen and tracked correctly by the system 101, more precisely by the optical device 102. The examiner helps the subject to put the headset correctly. So, the pupillary distance can be configured on the headset for example with the specific scroll wheel. Also, advantageously, the headset is set in the closest position to have the largest possible field of view.

The test realizes some or all of the following steps:
Vision check-up
Eye tracker calibration (optional)
Eye tracker validation (optional).
Right eye with free test
Left eye with free test
Right eye with choice test
Left eye with choice test
Right eye with Bielschowsky move (free test)
Left eye with Bielschowsky move (free test)

During the test, the subject can see different elements on both sides. The subject can check his field of view and determine if he correctly sees the different points to target. This allows the subject to check if the binocular vision works correctly at the projection distance (75 cm) and if the fusion is total (no diplopia).

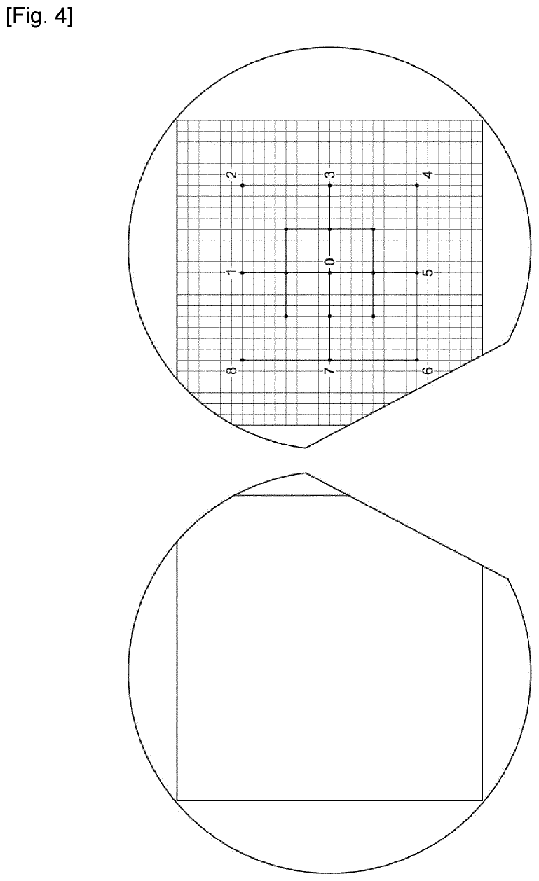

In an embodiment presented in FIG. 4, the first element is a Hess-Weiss grid (see right part of the figure) as seen in the optical device 102. This first element (in the example of FIG. 4 the Hess-Weiss grid) is displayed at a projection distance of accommodation distance of about 50 cm from the eye. The projection distance may vary from 50 cm to infinity (or more specifically to 2 m), depending on the configuration of the headset. Thus projection distance is typically around 75 cm. The projection distance may be varied by changing the optical system or the distance between the optical system and the screen. The projection distance depends on the optical device 102 and applies even if only one eye sees the displayed image. In comparison, the convergence distance applies only if both eyes see a similar displayed image shifted between the two eyes according to the convergence distance. The convergence distance can be varied by changing the displayed images as for standard virtual reality stereovision.

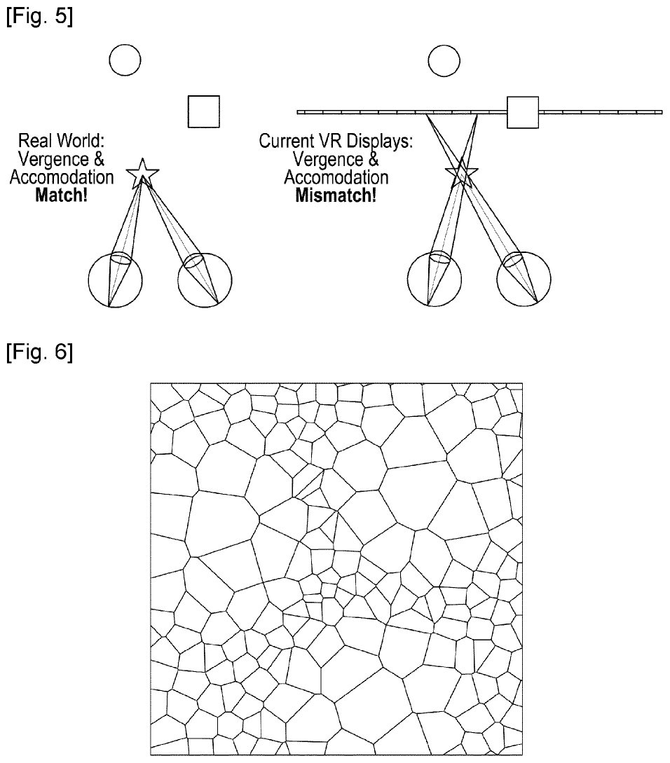

In real life, accommodation and vergence always match. Using the virtual reality headset this is not always the case. FIG. 5 represents a mismatch between the vergence and the accommodation. To be in the best conditions and match the accommodation and the vergence, the optical device 102 is configured to display a background at a convergence distance depending on an image projection distance of the optical device 102. For example, the convergence distance is between 80% and 120% of the image projection distance.

In other embodiments, the optical device 102 can present a mismatch accommodation-convergence condition to increase the difficulty of fusion and thus, increase the sensitivity of the test. Subjects with smaller binocular/motility vision problems are willing to have more difficulties to merge and succeed in the test with accommodation-convergence conflict.

As presented in FIG. 4, in the first step of Hess-Weiss experimentation (free test), the grid is displayed on one side (left or right), and a free white test is displayed at the other side. Binocular vision is dissociated in a more comfortable way than the real test, since rivalry between eyes is reduced: both eyes see a white background (while in the real test, the background is red for one eye and green for the other eye). We could even improve fusion/reduce rivalry by displaying only one point of the grid at a time (instead of the full grid).

In this first step the second element will be displayed on the part not displaying the Hess-Weiss grid (The Hess-Weiss being the first element) and the subject will have to move the second element to a specific location of the first element.

If a virtual reality headset is used, all elements displayed may be fixed on the screens whatever the inclination of the head (i.e. the display of the elements does not depend on the inclination of the head). This means that if the subject moves the head, the elements move also, following head movements since the subject is wearing the headset. This eliminates the need for a chinrest and allows the subject to be in a more natural position. It also allows being in a sitting or a standing up position. Furthermore, the location of the elements can be controlled, for example, the center of the Hess-Weiss grid is set and maintained precisely in front of the eye. Since the elements follow the head tilt, it is also easier to make the Bielschowsky test.

In an embodiment, the optical device 102 is configured to display the first image comprising a background and the second image comprising the same background. This background can be a Voronoi diagram or a dot grid or any 3D environment representing a particular scene, for example a virtual eye exam room, or any other abstract or artificial scene. The optical device 102 is configured to display the background at a distance depending on an image distance of the device. This distance is between 80% and 120% of the image distance. The FIG. 6 represents a Voronoi diagram.

In an embodiment, for the choice test, fusion is applied. For that, an additional element (a background) for the choice test (corresponding to the dot grid in the real Hess-Weiss test) is displayed on both eyes. In the real measurement test, the real environment is also part of the background and also helps to accommodate and fuse images because it works like a referential. To be closer to the real conditions, the virtual displaying may be a little bit different for the choice test step. The elements (for example the Hess-Weiss with a background dots grid) don't appear suddenly at the desired position for the test but progressively from far to near. The elements are projected at a far distance (about 3 meters) and get closer slowly until they are at the desired distance for the exam. The speed of this movement is calculated to be smooth and progressive. The closer the elements, the slower the speed (the speed is proportional to the proximity—inverse of the distance). This way helps the subject to accommodate and maintain fusion like in real life. Without this step, the subject can merge with offsets. With this movement, the merging & accommodation are better controlled.

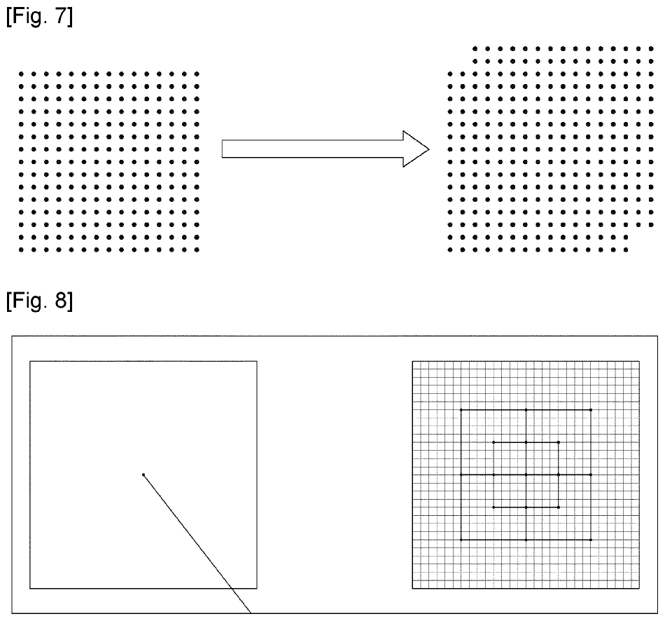

In other words, some backgrounds (like the Voronoi diagram) can be used to increase the binocular fusion. Especially when compared to a background of repeated black dots (see FIG. 7). When using repeated black dots, in case of visual deviations, the two images of the choice test are not aligned and it can cause diplopia on the edges. But at the center, the periodic spacings between each dot allows to compensate for the visual offset. Merging is possible. Nevertheless, in the virtual exam, some subjects still have misalignments.

FIG. 7 illustrates the above described difficulties arising when the background is constituted of a plurality of equidistant dots.

On one hand, we can increase this phenomena by removing the visibility of the frame contour, thus reducing cues to merge the image. The sensitivity of the test is then increased to detect vision problems that prevent from a correct fusion. On the other hand, we can reduce this phenomena and increase fusion. For that, we use another background without periodicity like Voronoi diagrams.

In an embodiment, the two images displayed respectively to the right eye and the left eye can have different colors and contrasts. One image can have a red background and the other can have a green background. We noticed that in the real test, the red and green backgrounds, for example obtained by filtering, don't have the same light absorption. Indeed, the green background is less absorbent than the red and it lets more light go through. For that, red and green backgrounds can be used in virtual exams with various contrasts. Red and green may be used to match the real Hess-Weiss test and can allow a better differentiation of each eye. In virtual, of course, as already mentioned, we could use a grid and a white background with different contrast on each eye to reduce rivalry and get a better comfort of fusion to merge images.

In an embodiment, the calculation module 103 or the optical device 102 can comprise a screen to give feedback to the examiner. This screen can implement a graphical user interface (GUI) that shows head angles, the depth of the grid, targeted positions, in live. The optical device 102 can comprise an orthographic camera that allows seeing what the subject sees through the headset. The orthographic camera allows the examiner to see both sides overlapping. It is not a problem if the exam is on a plane. If all objects are at the same distance, there is no need for a perspective projection. In that case, the orthographic projection is easier to interpret.

This GUI leads the examiner during the test. The protocol flow is easier to follow. All steps follow each other. The examiner just has to check the results on-screen step by step and validate with, for example, a key of the keyboard, for example, the space key, until the end of the exam. The GUI reduces or eliminates the need for manual steps.

In an embodiment, the optical device 102 comprises a joystick or a joypad that are examples of means 102-b to detect the movement. This joystick or joypad are movable by an hand of the subject. This movement of the joystick or joypad allows the movement of the second element. In this embodiment the selection means 102-c can be a switch or push-button, the switch or push-button being for example activatable by a finger of the subject. Joystick or joypad are less expensive than other detection means 102-b

In an embodiment, in the virtual scene, the joystick or joypad are seen with laser rays at the extremity. By moving the joystick or joypad with a first hand, the subject must target each point and validate. A validation button can for example be pressed with the second hand for not trembling with the main hand and to be more precise. He must validate each point in good order. Nine points appear step by step on the companion screen. Also, the subject must have a head angle between 10° and −10° to validate his target (i.e. we can set an acceptable threshold for head tilt at the time of measurement).

In FIG. 8, the subject sees the grid only on one side. The other eye sees the laser and a white square with the same dimensions as the grid. The eye which sees the grid is the fixating eye and the other one is the locating eye.

In an embodiment, the means 102-b to detect the movement is the eye tracker to detect a movement of the second eye. The optical device 102 is configured to move the second element according to the movement detected by the eye tracker. The means 102-b to detect the movement can be a binocular eye tracker. This binocular eye tracker helps the examiner to analyse the motility, and measure dissociated phoria simultaneously. The fixation differences between the two eyes are called the phoria when no fusion occurs (i.e. no background, no common elements between the 2 eyes) and are called the fixation disparity when fusion occurs, that is with some part (background in the choice test) to merge. The article “Heterophoria and fixation disparity: A review” published in Strabismus—2000, Vol. 8, No. 2, pp. 127-134 describes such problems of phoria and fixation disparity.

In the embodiment of the optical device 102 in which it comprises the eye tracker, the selection means 102-c can be a sensor configured to detect a blinking of the first eye or the second eye and to be activated after the blinking. The sensor can be the eye tracker used to detect the movement of the eye. When using the eye tracker, the blinking can be detected by the absence of the pupil.

In other embodiments, we can have a mix of detection means 102-b and selection means 102-c. For example, the eye tracker can be used with the validation button or the joystick or joypad can be used with the sensor configured to detect a blinking of one or two of the eyes of the subject.

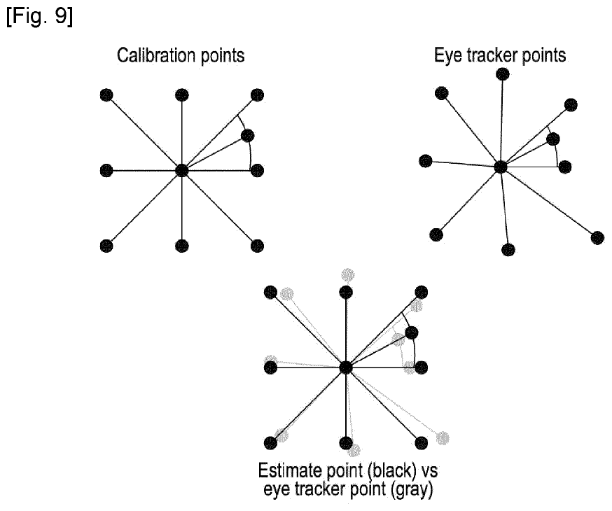

When using the eye tracker a calibration of this eye tracker can be realized to improve the accuracy of this eye tracker. In an embodiment of the calibration the subject looks at nine points that appear each in turn during 3 seconds. During the first second nothing is happening and 2 seconds of saving data. Data are averaged for each point. Then, eye tracker gazes are interpolated to correct raw data. The interpolation is done in a star referential format. We find in which sector from the referential the point is and how far it is of the central point. We also estimate the angle between the 2 edges of the sector to be able to find the position of the point after extrapolation. At the end of this calibration step, the two eye gazes are displayed on the examiner screen. They are represented by different colored points. The examiner can check in live if gazes are correct or not. If the result fits, he can validate and continue the exam. The FIG. 9 illustrates an example of calibration procedure.

The memory 103-a is configured to store a computer program comprising instructions which, when the program is executed by the processor 103-b, cause the calculation module 103 or the system 101 to carry out the steps of:
display the first element on the first image,
display the second element on the second image and to move this second element according to the movement of the part of the subject,
receive a selection by the subject;
once the selection is received, store a localisation of the second element in the second image and possibly a localisation of the first element in the first image.

These steps allow the examination of the ocular motility of the subject. In an embodiment, at the end of the exam, the examiner can check and interpret results in the exported file or with pictures. Therefore, misalignment phorias can be checked visually or by calculation. Other diagnoses are also possible: fixation disparity, tropias, any motility or binocular fusion problems. We could also realize calculating the motility diagnosis automatically with machine learning treatment.

In other words, using the embodiments of this disclosure we can diagnose and measure the state of binocular vision and ocular motility, with a test that is easy to explain, accurate, fast and easy to interpret by the examiner. These embodiments allow realizing a more «natural» Hess/Weiss measurement, more comfortable for the subject, using for example a virtual reality headset, allowing the subject to have a free head posture (no chinrest) even for Bielschowsky testing, with less eye rivalry. By with less eye rivalry, we mean that this is not mandatory to use red image vs green image. The virtual reality headset allows the subject to display a fixed image in the world frame of reference, to measure the position of the head of the subject compared to a target. This measure can be realized using an Inertial Measurement Unit IMU integrated in the optical device 102 and/or the eye tracker to measure the direction of gaze. We can also use a virtual laser controlled by the joypad to allow the subject to show where he looks.

The virtual reality headset can also comprise lenses that can be changed. By changing the lenses in the virtual reality headset we can change the accommodation plan in the test. We can also change the convergence plan with the 3D capabilities of the headset accordingly or separately from the accommodation.

Thus, the examiner will be able to run the test in a lot of various conditions, he will have the Hess Weiss «Ergorame». He can use this test at different distances, this one being representative of an ergorama for progressive lenses, and he can also simulate disparities, in order to carry out measurements in line with the use of a Progressive Addition Lenses (PAL). Ergorama is a function associating to each gaze direction the usual distance of an object point. He can also simulate the progressive lens worn in order to check the suitability of the chosen design with the disparity measurements and he can even integrate a smooth pursuit test with a moving target.

Thanks to the headset, the examiner will have new possibilities to measure the troubles of binocular vision in far, near and intermediate distances and for different gazes directions, with or without the glasses of the subject (to help the decision of prescription of binocular vision changes). He would have greater distances as well as being able to perform tasks closer to reality (use of computer, reading, television . . . ) to determine ocular motility in everyday situations and know what causes visual fatigue and adaptation problems to the optical prescription (SV, PALs . . . ). SV means Single Vision lenses (ie as opposed to multiple power lenses such as bifocals or progressive lenses).

The software of the calculation module can be configured to display a simple graph to give the diagnosis to the examiner and display 3D explanations to the subject. The software can also display simple explanations of the tasks during the measurements.

The system 101 and more precisely the computational module 103 can also determine which ocular muscle of the subject does not function well.

1. A system to assess an ocular motility of a subject comprising:
a device to assess an ocular motility of a subject, the device having at least one screen configured to display a first image to a first eye of the subject and a second image to a second eye of the subject,
the first image having a first element that is fixed and the second image having a second element that moves according to a movement of the subject,
calculation circuitry having a memory and a processor, the calculation circuitry being configured to:
display the first element on the first image,
display the second element on the second image and to move this second element according to the movement of part of the subject,
receive a selection by the subject, and
once the selection is received, store a localization of the second element in the second image and a localization of the first element in the first image.

2. The system according to claim 1, wherein the device further includes two screens, a first of the two screens being configured to display the first image and a second of the two screens being configured to display the second image.

3. The system according to claim 1, wherein the device further includes a single screen and a filter, the single screen and the filter being configured to display the first image to the first eye and the second image to the second eye.

4. The system according to claim 1, wherein the device further includes circuitry to detect the movement of a part of the subject, and
wherein the device is further configured to move the second element according to the detected movement of the part of the subject.

5. The system according to claim 4, wherein the circuitry to detect the movement is an eye tracker to detect a movement of the second eye, and
wherein the device is configured to move the second element according to the movement detected by the eye tracker.

6. The system according to claim 4, wherein the circuitry to detect the movement is a joypad movable by a hand of the subject, and
wherein the device is configured to move the second element according to the movement of the joypad.

7. The system according to claim 1, wherein the device further includes selection circuitry configured to be activated by the subject.

8. The system according to claim 7, wherein the selection circuitry is a switch or push-button, and
wherein the switch or push-button is activatable by a finger of a first hand of the subject, the first hand being different from a second hand of the subject used to activate circuitry to detect the movement.

9. The system according to claim 7, wherein the selection circuitry is a sensor configured to detect a blinking of the first eye or the second eye and to be activated after the blinking.

10. The system according to claim 1, wherein the device is further configured to display a first image having a background and the second image having the background.

11. The system according to claim 10, wherein the background is a Voronoi diagram.

12. The system according to claim 10, wherein the device is configured to display the background at a distance depending on an image distance of the device.

13. The system according to claim 12, wherein the distance is between 80% and 120% of the image distance.

14. The system according to claim 1, wherein the device is configured to display the first image and the second image independently of an orientation of a head of the subject.

15. A computer implemented method to assess an ocular motility of a subject, the method comprising:
displaying to a first eye of the subject a first image having a first element;
displaying to a second eye of the subject a second image having a second element and moving the second element according to a movement of a part of the subject;
receiving a selection by the subject;
once the selection is received, storing a localization of the second element in the second image and a localization of the first element in the first image.

16. The system according to claim 3, wherein the filter is a polarizing filter.
